(19) United States
(12) Patent Application Publication
(10) Pub. No.: US 2015/0040641 A1
(43) Pub. Date: Feb. 12, 2015

(54) PASSIVELY COOLED GAS DETECTOR

(71) Applicant: Crowcon Detection Instruments Limited, Abingdon, Oxfordshire (GB)

(72) Inventor: Roger Hutton, Abingdon (GB)

(21) Appl. No.: 14/385,214

(22) PCT Filed: Mar. 14, 2013
(86) PCT No.: PCT/GB2013/050629

§ 371 Date: Sep. 15, 2014

(30) Foreign Application Priority Data
Mar. 15, 2012 (GB) 1204573.8

(51) Int. Cl. G01N 33/00

(52) U.S. Cl. CPC G01N 33/0016
USPC 73/23.21

(57) ABSTRACT

A gas detector assembly which is at least in part passively cooled comprising; a thermal chimney comprising a first heat collecting area in fluid communication with a ventilation area; a gas sensing device placed at least in part in the thermal chimney, such that in use the first heat collecting area is heated and a temperature gradient between the heat collecting area and ventilation area is established providing a passive cooling flow of gas over at least part of the gas sensing device placed in the thermal chimney.

This application is a United States national phase application based on PCT/GB2013/050629 filed Mar. 14, 2013 which claims the benefit of British Patent Application No. GB 1204573.8, filed Mar. 15, 2012. The entire disclosures of the above applications are hereby incorporated herein by reference.

### FIELD OF INVENTION

The present invention relates to gas detectors. In particular, it relates to the use of thermal chimneys in order to increase the gas flow over gas sensing devices.

### BACKGROUND TO THE INVENTION

It is known that the performance of gas sensing devices is dependent upon the ambient temperature and operating conditions. In particular, extreme temperatures can affect the operation of a wide-range of sensor types, including electrochemical cells, pellistors, IR sensors and luminescence-based sensors. For example, in the case of electrochemical cells, the sensitivity to an individual gas can be greatly affected by temperature. As the operating temperature is increased, the sensor may become more sensitive to a particular target gas that is desirably measured.

Another known effect that results from operating at higher temperatures is that component lifetimes are reduced. This can be attributed to various thermally-based degradation issues, including, for example, electrolyte evaporation, which can be enhanced by operating at an elevated temperature, resulting in reduced lifetime. Prolonged operation at elevated temperatures may result in evaporation of the electrolyte and subsequent sensor failure. This leads to increased costs in replacement and maintenance of the devices.

The effects highlighted above may become more relevant dependent upon the location in which the devices are being used. For example, gas sensors are often used in the Middle or Far East, where conditions are relatively extreme in terms of temperature and humidity. The sensors are often placed in environments which are subject to radiation from the sun, which causes the sensors to become hotter which depending on the level of heating experienced may subsequently affect their performance.

In addition to external sources of radiation, electrical components used in gas-sensing devices can be a further source of heat that can contribute to increased temperature.

In order to mitigate for increased heating, the gas sensing device may be actively cooled using, for example, a peltier device, air-conditioning system, etc. However such cooling systems are costly to produce and require power sources, which can result in increased production, running and maintenance costs.

In order to mitigate for at least one of the problems above, there is provided a passively cooled gas detector, comprising a thermal chimney, having a first heat collecting area in fluid communication with a ventilation area and a gas sensing device placed at least in part in the ventilation area, such that in use, when the first heat collecting area is heated, a convection flow between the heat collecting area and ventilation area is established, and provides a cooling flow over at least part of the gas sensing device. An advantage of this invention is that cooling is provided when it is most needed, i.e. when the detector is exposed to the sun. The invention passively maintains a steady temperature that improves sensor lifetime and reduces calibration and maintenance cycles.

Such an assembly advantageously does not rely on an internally generated power source. The passive operation may result in reduced costs, since it provides a cooling mechanism without complicated equipment that requires its own power supply. Such an arrangement may reduce running and maintenance expenditure. Also, by shaping the top of the chimney with a ‘hat’, gas will be drawn through by way of the Bernoulli effect.

Advantageously it has been realised that a further benefit of such a system is that it enables the gas detector to sample gas from a wide area as opposed to a single point. Typically, gas sensors rely on natural diffusion of gases from the point of release to the point of detection at the gas sensor itself, however, this process is dependent on effects such as Brownian motion and are slow. As the timescale over which is a target gas is sampled is typically short the sensor will effectively only sample the gas in the immediate vicinity of the sensor and acts as a “point sensor”. In use, the assembly described above can increase gas flow past the gas sensor, which results in a larger volume of gas being sampled, compared with a conventional point sensor that is reliant on the natural flow of surrounding gas. Advantageously the positive gas flow past the sensor enables an increased effective volume of gas to be measured compared to prior art “point” system.

The assembly allows for the protection of the gas sensing device from high ambient temperatures, and the effects of solar radiation, without the need for an expensive cooling system. This results in more stable measurements without extra costs.

In accordance with an aspect of the invention there is provided a gas detector assembly which is at least in part passively cooled comprising: a thermal chimney comprising a first heat collecting area in fluid communication with a ventilation area; a gas sensing device placed at least in part in the thermal chimney, such that in use the first heat collecting area is heated and a temperature gradient between the heat collecting area and ventilation area is established providing a passive cooling flow of gas over at least part of the gas sensing device placed in the thermal chimney.

In accordance with another aspect of the invention, there is provided a method of assembling the passively cooled gas detector, wherein the thermal chimney is connectable to the gas sensing device.

Further aspects of the invention will be apparent from the description and the claims.

### BRIEF DESCRIPTION OF THE FIGURES

Embodiments of the invention are now described, by way of example only, with reference to the accompanying drawings in which:

FIG. 1 shows an assembly comprising a thermal chimney and a gas sensing device, according to one aspect of the invention;

FIG. 2 shows an assembly comprising a thermal chimney, a gas sensing device and a connector, according to one aspect of the invention;

FIG. 3 illustrates an assembly comprising a thermal chimney and a gas sensing device, whereby the assembly is mounted in such a way that the air in the thermal chimney is heated by solar radiation and the gas sensing device is shielded from the solar radiation; and

FIG. 4 illustrates an assembly comprising a thermal chimney and a gas sensing device, whereby the cross-sectional area of the thermal chimney is greater at the entrance and exit, compared with the central part.

### DETAILED DESCRIPTION OF AN EMBODIMENT

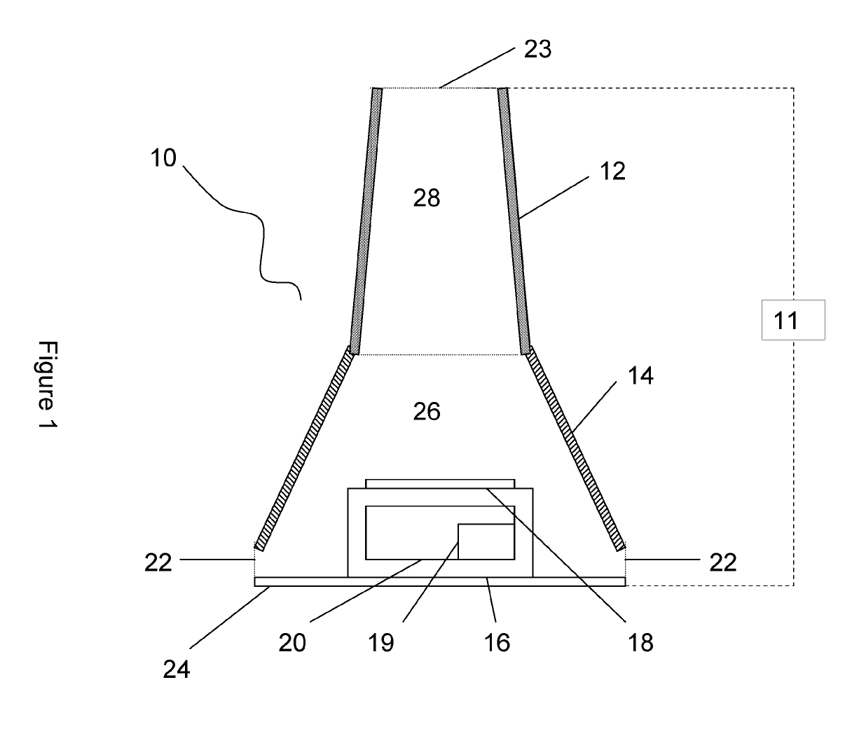

FIG. 1 is a schematic of a gas detector assembly of a thermal chimney and a gas sensing device according to one aspect of the invention.

In FIG. 1, there is shown a gas detector assembly 10 comprising a gas sensing device 16 and a thermal chimney 11. The gas sensing device 16 comprises: a gas sensor 18; a power supply 19; and integrated circuits 20. The thermal chimney 11 comprises: heat collecting walls 12, connected to ventilation area walls 14 and a base 24. There are a plurality of vents 22 in the ventilation area walls 14. The ventilation area walls 14, along with the base 24, define a ventilation area 26. The heat collecting walls 12, along with the chimney exit 23 define a heat collecting area 28. The ventilation area 26 is in fluid communication with the heat collecting area 28. The device 16 is integrated in to the ventilation area 26.

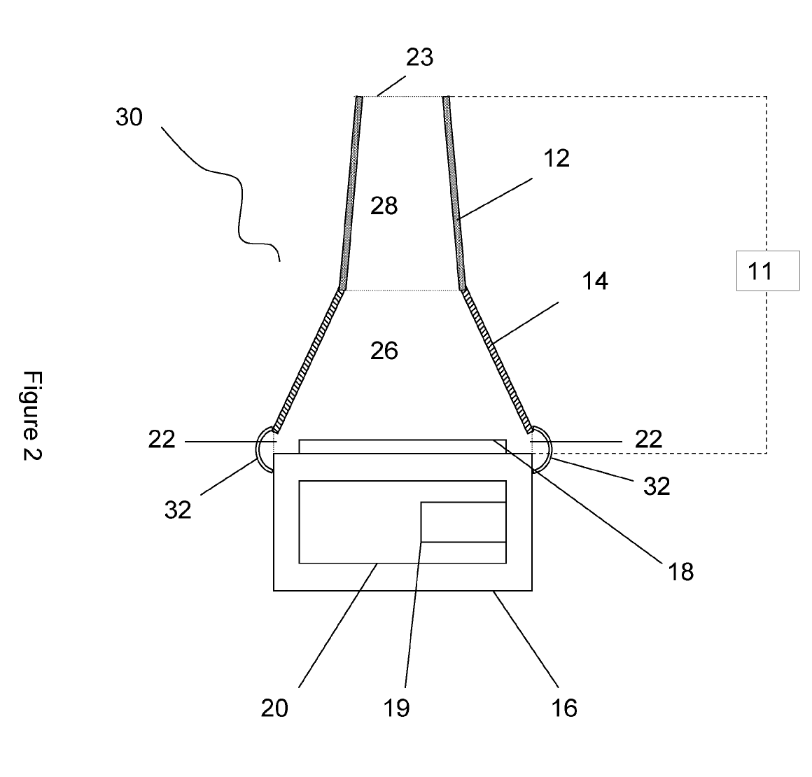

The heat collecting walls 12 are made from a material which preferentially absorbs radiation from an external source such as the sun. In use the sun causes the gas in the heat collecting area 28 to heat. Preferably the ventilation area walls 14 are made from a material which preferentially reflects radiation from the sun, allowing for a temperature differential to be created between the gas in the heat collecting area 28 and the ventilation area 26. The temperature outside of the thermal chimney 11 is preferably cooler than inside the heat collecting area 28. Subsequently, the gas heated in the heat collecting area 28 rises out of the thermal chimney 11 due to the creation of a convection current and is replaced with relatively cooler gas from the ventilation area 26. The gas in the ventilation area 26 is then replaced by a supply of gas from outside of the thermal chimney 11, which enters into the thermal chimney 11 through a plurality of vents 22. The replacement gas in the heat collecting area 28 is then heated and the cycle continues, with fresh gas being drawn through the plurality of vents 22, through the ventilation area 26, into the heat collecting area 28, before being expelled through the chimney exit 23. The process is a continuous one, with the overall effect being that there is a steady flow of gas through the thermal chimney 11. As the gas sensing device 16 is placed within the ventilation area 26 the flow of gas passes over the device 16 causing it to cool. In other embodiments for example FIG. 2 the gas sensing device 16 is placed partly within the ventilation area 26, and the part of the device in the ventilation area 26 experiences the gas flow, thereby cooling the device.

The effect of the gas passing the gas sensing device 16 is manifold; it increases heat transfer away from the gas sensing device 16 due to the natural tendency of the warmer object to obtain thermal equilibrium with the surroundings. Since the nature of the gas at the gas sensing device 16 is transient and it is continuously being replenished with cooler gas, the overall effect is that there is an increased rate of heat transfer away from the gas sensing device 16 through the thermal chimney 11, until thermal equilibrium is reached. A further effect of having a continuous flow of gas past a gas sensing device 16 is that the temperature of the gas sensing device 16 is stabilised. This helps ensure that the device 16 produces accurate readings.

Preferably the ventilation area walls 14 are made from a material which preferentially reflects solar radiation, thereby reducing the amount of radiation that can penetrate into the ventilation area 26 directly, or through absorption in the ventilation area walls 14 and re-radiation in to the ventilation area, therefore more efficiently forming a temperature differential between the ventilation area 26 and the heat collecting area 28 in the thermal chimney 11. The ventilation area walls 14 are made from white painted metal though ceramic, plastic or a double skin structure may be used.

Preferably the heat collecting walls 12 are at made from material that absorbs radiation more efficiently than the ventilation area walls 14. Such material efficiently absorbs incident radiation, in particular solar radiation, including infra-red, visible and ultra-violet wavelengths. The resultant effect is that the heat collecting walls 12 efficiently absorb the incident radiation (in particular the solar radiation) and re-emit the energy into the heat collecting area 28, causing the gas within the heat collecting area 28 to warm up. The heat collecting walls 12 are made from black metal though clear materials may be used.

Preferably the heat collecting walls 12 are made from a material that has a higher thermal capacity than the ventilation area walls 14. When the heat collecting walls 12 are subjected to solar radiation, as the walls 12 are made from a material with higher thermal capacity the heat collecting walls 12, store more heat energy than the ventilation area walls 14. Subsequently, the heat transfer from the heat collecting walls 12 into the heat collecting area 28 is more efficient than the heat transfer from the ventilation area walls 14 into the ventilation area 26. Advantageously, as material with a high thermal capacity is used more energy is stored in the walls 12 and subsequently reemitted into the heat collecting area 28 thereby warming the gas present in the heat collecting area 28. This advantageously allows the cooling flow created by the thermal chimney to continue, for a period of time, in the absence of solar radiation. For example after sunset, the difference in thermal capacity between the heat collecting walls 12 and the ventilation area walls 14 means that the temperature differential that was created between the heat collecting area 28 and the ventilation area 26 can be maintained. Heat energy is retained in the heat collecting walls 12 and released over an extended period of time thereby heating the gas present in the heat collecting area 18. The consequence of this is that a convection flow that has been created by gas in the ventilation space 26 replacing gas in the heat collecting area 28 will continue in the absence of the solar radiation and the gas sensing device 16 will continue to be cooled. There will thus be more stable sampling of the gas whilst the ambient conditions may still be warm.

The gas sensing device 16 is a known commercially available gas detector. In such devices the power supply 19 and integrated circuits 20 also contribute to the heating of the device and surrounding environment. The gas flow in the ventilation area 26 beneficially also provides a cooling flow over the heat producing elements of the supply thereby decreasing the temperature of the device 16.

The cooling flow experienced by a sensor placed, at least in part, in the thermal chimney will be in part determined by the cross-sectional area of the part of the thermal chimney 11 in which the sensor is placed. The rate of flow of gas through the entire thermal chimney 11 will ultimately depend on the temperature gradient between the heat collecting area 28 and the ventilation area 26, as the higher the temperature gradient, the greater the convection current produced. The rate of cooling flow within a specific part of the thermal chimney 11 is dependent on the cross-sectional area due to conservation of flow. By reducing the cross-sectional area of a specific part of the chimney, choke points can be created that increase the rate of gas flowing through that region. Therefore by placing the sensor, or part of the sensor to be cooled, in an area which has smaller cross-sectional area, the cooling effect can be accentuated. Optionally, the chimney 11 is shaped with a ‘hat’ such that gas will be drawn through by way of the Bernoulli effect.

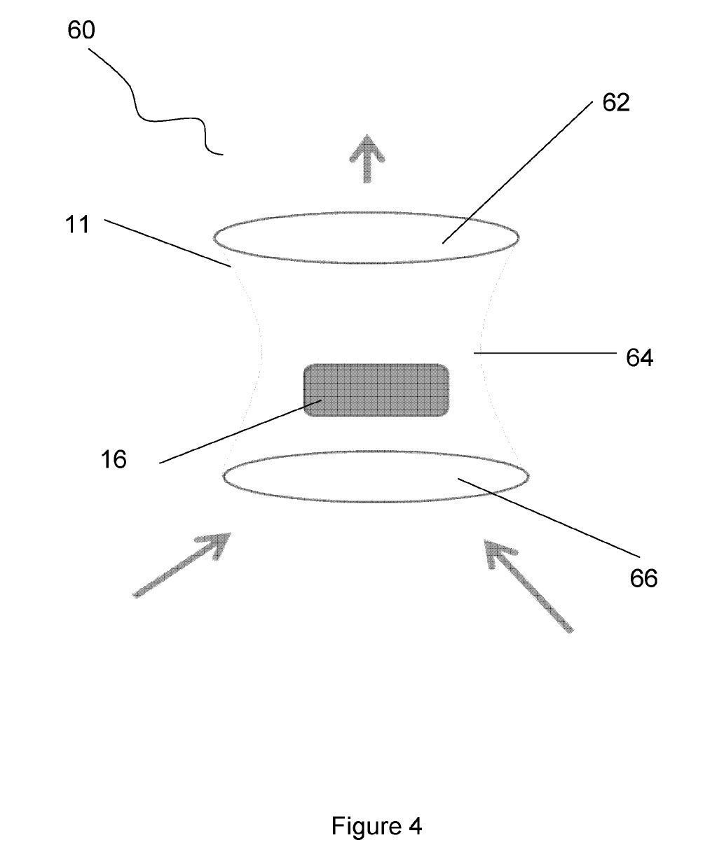

FIG. 4 shows an example of a thermal chimney 11 in which a gas sensing device 16 is placed. The first cross-sectional area 62, corresponding to the chimney exit 23 is larger than the second cross-sectional area 64, in the middle of the thermal chimney 11. The third cross-sectional area 66, corresponding to the vent 22 for the inlet of gas, is larger than the second cross-sectional area 64. Consequently, the passage of gas through the thermal chimney 11 varies as it passes from the third cross-sectional area 66 to the first cross-sectional area 62. Cross-sectional area 64 therefore acts as a choke point. At the choke point, the flow of gas through the thermal chimney goes from a wider cross-sectional area 66 to a narrower cross-sectional area 64, there is a lower pressure at the choke point. This results in an increased cooling effect at the choke point, corresponding to the smaller cross-sectional area 64. The cross-sectional area along the length of the thermal chimney 11 can be selected to create areas of increased, or decreased flow. Furthermore, the gas sensing device 16 may be placed in areas of increased or decreased cross section depending on the level of cooling flow desired.

Optionally, the heat collecting walls 12 comprises a plurality of sections made from materials with different material properties. This can be used to control the rate of gas flow through the thermal chimney as well as to create areas of increasingly homogenous temperatures.

Optionally, the heat collecting walls 12 have a plurality of layers. By increasing the number of layers, it is possible to partially insulate the gas in the heat collecting area 28 from the gas outside of the thermal chimney 11. Consequently, heat loss from the gas in the heat collecting area 28 through the heat collecting walls 12 is reduced and the heat differential between the heat collecting area 28 and the ventilation area 26 is established more efficiently. In an example of the invention, a plurality of layers of the heat collecting walls 12 are partially transparent to solar radiation and thus the gas in the heat collecting area 28 heats up more efficiently due to the greenhouse effect.

Optionally, the ventilation area walls 14 have a plurality of layers. By increasing the number of layers, the gas in the ventilation area 26 is insulated from the ambient temperature, which contributes to the thermal stability of the gas sensing device 16, which in turn leads to more accurate sampling of gas.

Further benefits may occur from using a plurality of layers in the heat collecting 14 and ventilation area 12 walls, for example the chimneys may consequently be more durable and sturdier.

Optionally, there are vents 22 in the base 24 of the thermal chimney 11, which can be used to replace vents in the ventilation area walls 14, or in addition to them. The vents can be configured in order to optimise gas flow through the thermal chimney 11 by varying their number, size and position. Optionally, the chimney is ‘hat’ shaped such that gas is drawn through the chimney by way of the Bernoulli effect.

Advantageously the present configuration of the gas detector assembly allows for the gas to be sampled from a wider volume. Conventional gas detectors have relatively short timescales for sampling gases and therefore the sensors only effectively detect gases that are in the immediate vicinity of the sensor, therefore these sensors act as “point sensors”. By creating a positive flow of gas over a gas sensor, the rate of gas passing the sensor increases, resulting in an increased volume of gas being sampled. This means that there is an effective increase in the volume of gas that is being sampled, compared with a conventional “point sensor”. This is beneficial in, for example, a situation where a sensor is being used to signal an alarm upon the detection of the presence of a toxic gas. In ordinary circumstances, the detection of the toxic gas would be dependent on the natural diffusion of gas through effects such as Brownian motion. These processes are relatively slow and there may be a considerable delay between a toxic gas escaping at a leak point and being detected at the sensor, depending upon the position of the sensor. By creating a positive airflow past the sensor, using a thermal chimney, the passage of gas is increased, the effective size of area measured is increased and the detection is faster, This can result in a faster, safer system, with fewer sensors needed in the same target area and a more stable reading.

FIG. 2 is a schematic of a gas detector assembly of a thermal chimney connected to a gas sensing device according to one aspect of the invention where the thermal chimney is retrofitted to an existing gas sensing device.

In FIG. 2, there is shown a gas detector assembly 30 comprising a gas sensing device 16, a thermal chimney 11 and connectors 32. In an example of the invention the gas sensing device 16 comprises: a gas sensor 18, a power supply 19 and integrated circuits 20. The thermal chimney 11 comprises: heat collecting walls 12, connected to ventilation area walls 14. There is a plurality of vents 22 in the ventilation area walls 14. The ventilation area walls 14 are connected to the gas sensing device 16 by a plurality of connectors 32, which are preferably mechanical connectors. In further embodiments other types of connectors are used, for example magnetic connectors, chemically-fixed connectors, suction-fixed connectors etc. The ventilation area walls 14, along with the gas sensing device 16, define the ventilation area 26. The heat collecting walls 12, along with the chimney exit 23, define the heat collecting area 28. The ventilation area 26 is in fluid communication with the heat collecting area 28. In an example of the invention, the gas sensing device 16 can be detached and reattached to the thermal chimney 11.

In such an embodiment the detector is located under the ventilation area walls 14 and therefore is, at least in part, in the ventilation area 26. Therefore at least part of the detector 10 experiences the cooling flow.

In one embodiment, thermal chimney 11 is retrofitted to an existing gas sensing device 16. The gas sensing device 16 is a conventional commercially manufactured device. In an example, the gas sensing device 16 forms the base of the thermal chimney 11, where the thermal chimney 11 is connected to the gas sensing device 16 using a plurality of connectors 32, such that the ventilation area walls 14 are sealingly connected to the gas sensing device 1 and in operation, gas is drawn through the vents 22 in the ventilation area walls 14, over the gas sensing device 16 and through the thermal chimney 11.

In further examples the gas sensing device partially forms the base of the thermal chimney 11, where there is a gap between the ventilation area walls 14 and the gas sensing device 16, such that the gap between the two acts as the vents 22, through which the gas is drawn into the thermal chimney 11.

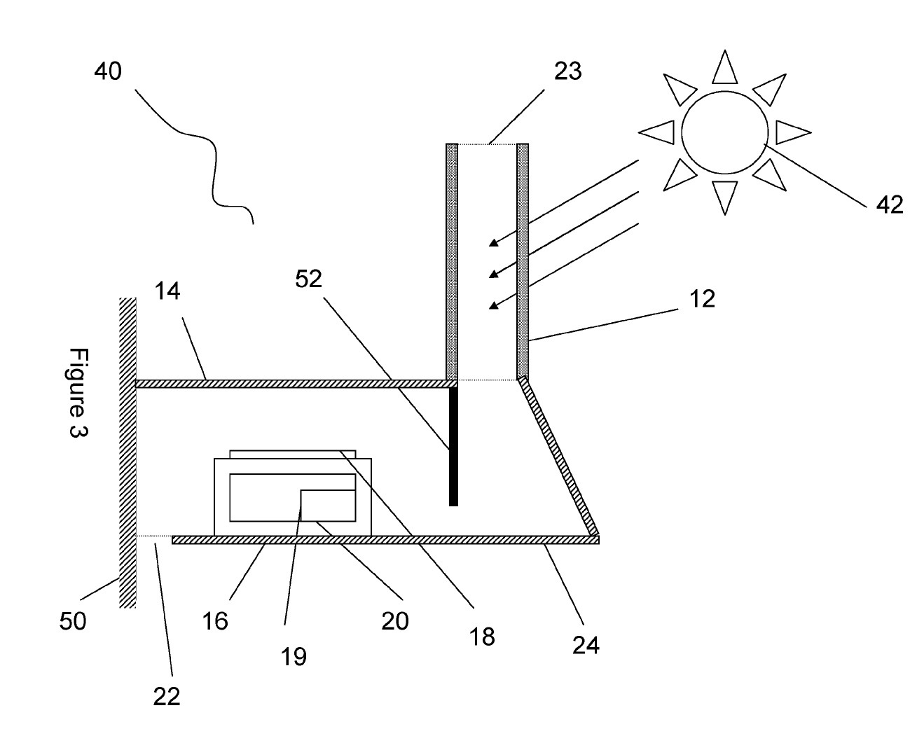

FIG. 3 is a schematic of a mounted gas detector assembly comprising a thermal chimney and a gas sensing device, whereby the assembly is mounted in such a way that the gas in the thermal chimney is partially heated by solar radiation and the gas sensing device is shielded from solar radiation. Such devices are mountable onto solid surfaces such as walls.

In FIG. 3, there is shown a gas detector assembly 40 comprising a gas sensing device 16 and a thermal chimney 11. In an example of the invention the gas sensing device 16 comprises: a gas sensor 18, a power supply 19 and integrated circuits 20. The thermal chimney 11 comprises: heat collecting walls 12, connected to ventilation area walls 14 and a base 24. There is a plurality of vents 22 in the base 24. The ventilation area walls 14, along with the base 24, define the ventilation area 26. The heat collecting walls 12, along with the chimney exit 23 define the heat collecting area. The ventilation area 26 is in fluid communication with the heat collecting area 28. The thermal chimney 11 is attached to a wall 50 such that the heat collecting walls are heated by solar radiation 42. The ventilation area 26 is insulated from the solar radiation 42 by insulating wall 52.

In use, the apparatus is assembled such that part of the thermal chimney 11 has the heat collecting walls 12 suitably positioned to absorb solar radiation 42, throughout daylight hours, as far as is practical. The ventilation area 26 is shielded from the solar radiation 42 and insulated from the heat collecting area 28 by insulating wall 52 and due to the temperature differential formed between the heat collecting area 28 and the ventilation area 26, gas is drawn in through the vents 22, passing by the gas sensing device 16 and exiting through the thermal chimney exit 23.

In another example, the thermal chimney 11 has variably deformable geometry, such that the form and size of any of the heat collecting walls 12, ventilation area 14, base 24 and vents 22 can be changed in order to alter the gas flow rate and uniformity of gas flow through the thermal chimney. The ventilation area walls 14 may be constructed from an sheet made from elastic and reflective material that can be stretched over a series of rings of increasing diameter that are positioned increasingly further from the heat collecting area 28, thus altering the effective cross-section of the ventilation area 26 along the length of the chimney 11. The heat collecting walls 12 may consist of a telescopic portion. The extension and retraction of the telescopic portion can result in a variation in the size of the heat collecting area 28, which can in turn be used to vary the rate of gas flow through the thermal chimney 11.

In another example, the thermal chimney 11 is configured such that the uniformity and rate of flow of gas through the thermal chimney enables the sampling of gas from a targeted volume and at a targeted rate.

In the present examples, a gas sensing device 16 is an electrochemical cell. It contains a gas sensor 18 that is integrated into the gas sensing device 16. It also contains a power supply 19 and integrated circuits 20. In further examples, a gas sensing device 16 is an IR sensor or luminescence based sensor.

In a further example, the gas sensing device 16 is connected to a remote system for monitoring gas samples. The system can be used to display and process samples of gases, or to signal an alarm, or to engage a process upon receipt of a reading.

In the illustrated examples, one gas sensor 18 is shown per thermal chimney 11. However, in further examples, a plurality of sensors 18 is associated with the beneficial effects of operating in conjunction with a thermal chimney 11, i.e. there can be more than one gas sensor 18 in a thermal chimney 11.

In the illustrated examples the gas sensing device 16 is placed either in the thermal chimney 11, or forms the base of the ventilation area 26. As the sensor defines the base of the ventilation area it is at least in part in the thermal chimney. In other examples (not shown), the thermal chimney 11 is placed above the gas sensing device 16 on stilts, in order to achieve the previously described cooling effect. In further examples (not shown) the thermal chimney 11 is suspended above a gas sensing device 16 by virtue of a frame etc. In these examples, as with the example described above with respect to FIG. 2, the detector is therefore positioned under the ventilation area walls 14 and therefore is, at least in part, in the ventilation 26. Therefore at least part of the detector 10 experiences the cooling flow from the thermal chimney.

In the illustrated examples, the chimney exit 23 is exposed the surroundings, in further examples, the chimney is shaped with a ‘hat’ such that gas will be drawn through by way of the Bernoulli effect.

The above examples refer to passive cooling, whereby a cooling effect is produced without the need for a power supply. This reduces running and maintenance costs as well as construction costs.

In further examples (not shown), the passive cooling system described above is supplemented by using a further powered (non-passive) cooling system. The powered cooling system provides a further cooling effect to the gas detector 10, which works in conjunction with the passive cooling system described above. In an example, electrical heat sources are used to cause convective transport in the chimney. Therefore, the electrical heat sources are placed in the chimney and work in addition to the heat collecting walls 12 to heat the heat collecting area 28. Optionally, one or more fans are placed in the ventilation area 28 and/or heat collecting area 26 to force mass transport to provide a supplemental cooling effect. In a further embodiment the fans are placed outside of the thermal chimney 11 and are configured to blow gas into the vents 22 and to draw gas from the exit 23, thereby supplementing the gas flow through the thermal chimney 11 due to the passive cooling system previously described. In a further embodiment, the fans are at least partially placed in the thermal chimney 11, in order to supplement the flow of gas through the thermal chimney 11.

The supplemental cooling effect can be used to aid the stabilisation of the temperature of the gas sensor and hence its output. It can further be used to supplement the cooling and stabilisation effect of the thermal chimney 11 in the absence of solar radiation, for example, during the night, or in due to cloud cover.

1-22. (canceled)

23. A gas detector assembly which is at least in part passively cooled, the gas detector assembly comprising:
a thermal chimney comprising a first solar heat collecting area in fluid communication with a ventilation area; and
a gas sensing device, at least part of the gas sensing device placed in the thermal chimney, wherein the first solar heat collecting area is heated during use and a temperature gradient is established between the heat collecting area and the ventilation area, thereby creating a passive positive airflow of a gas over the at least part of the gas sensing device placed in the thermal chimney, thereby providing an effective increase in a volume of the gas sampled by the gas sensing device.

24. The assembly of claim 23, wherein the gas sensing device is configured to sample the gas over an increased volume, due to the flow of gas through the thermal chimney.

25. The assembly of claim 23, wherein the first solar heat collecting area has one or more walls.

26. The assembly of claim 25, wherein the ventilation area has one or more walls.

27. The assembly of claim 23, wherein the first solar heat collecting area is at least partially produced from a material which has a higher thermal capacity than a material the ventilation area is produced from.

28. The assembly of claim 23, wherein the first solar heat collecting area is at least partially produced from a more electromagnetic-radiation absorbing material than a material the ventilation area is produced from.

29. The assembly of claim 23, wherein the ventilation area is at least partially produced from a more electromagnetic-radiation reflecting material than a material the first solar heat collecting area is produced from.

30. The assembly of claim 23, wherein at least part of the first solar heat collecting area is configured to be transparent to a type of electromagnetic-radiation, including infra-red, visible, and ultra-violet wavelengths.

31. The assembly of claim 23, wherein the thermal chimney has a variable cross-section, and wherein a rate of flow of the gas through the thermal chimney is controllable.

32. The assembly of claim 23, wherein the thermal chimney is configured such that a form and a ratio of the first solar heat collecting area to the ventilation area is such that gas passes through at a desired sampling rate.

33. The assembly of claim 26, wherein a thermal capacity of the one or more walls of the first solar heat collecting area is higher than a thermal capacity of the one or more walls of the ventilation area, and wherein the first solar heat collecting area can store energy that is released, heating the first solar heat collecting area to obtain or maintain the passive positive airflow through the chimney.

34. The assembly of claim 33, wherein the passive positive airflow is controlled in order to control the temperature of the device.

35. The assembly of claim 33, further comprising an electrical heat source disposed in the thermal chimney, the electrical heat source configured to cause convective transport in the thermal chimney supplementing the passive positive airflow of gas.

36. The assembly of claim 33, further comprising a fan configured to force mass transport of the gas supplementing the passive positive airflow of gas.

37. The assembly of claim 23, wherein the gas sensing device is an integral part of the thermal chimney.

38. The assembly of claim 23, wherein the gas sensing device is placed wholly in the ventilation area.

39. The assembly of claim 23, wherein the gas sensing device is placed at least partly in the first solar heat collecting area,

40. The assembly of claim 25, wherein the assembly is adapted to be mounted on a wall wherein the one or more walls of the first solar heat collecting area are heated by solar radiation.

41. The assembly of claim 23, wherein the thermal chimney is connectable to the gas sensing device.

42. The assembly of claim 41, wherein the gas sensing device can be detached from and reattached to the thermal chimney.
